(19) United States
(12) Patent Application Publication
(10) Pub. No.: US 2006/0281099 A1
(43) Pub. Date: Dec. 14, 2006

(54) Q-melt for polymorphism detection

(75) Inventors: John W. Breneman, Brentwood, CA (US); Nancy McKinney, Del Mar, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Bio-Rad Laboratories, Inc., Hercules, CA (US)

(21) Appl. No.: 11/153,236

(22) Filed: Jun. 14, 2005

(51) Int. Cl. C12Q 1/68; C12P 19/34

(52) U.S. Cl. 435/6; 435/91.2

(57) ABSTRACT

The invention provides a new method of determining the presence of one or more polymorphic sites in a target nucleic acid sequence. The method comprises determining changes in the melting profile of an amplified product where the amplified product was amplified with a primer pair where one of the primers is labeled, e.g., with a fluorescent label. The method employs a probe that is labeled with a label that interacts with the label on the primer, e.g., a quencher that quenches the fluorescent label on the primer, such that the signal is quenched when the probe and primer are in close proximity. A change in the melting profile of an amplified product in comparison to a control reflects the presence of the polymorphic site.

### BACKGROUND OF THE INVENTION

This invention relates to a new method of detecting polymorphisms in a target nucleic acid sequence. The detection of polymorphisms is useful, e.g., in determining the genotype of a patient. Conventional melt curve analysis for the detection of polymorphisms is structured such that a change in fluorescence is generated when an oligonucleotide(s) is hybridized to a target nucleic acid that is polymorphic at a position that is in the region targeted by the probe. For example, one such method involves a dual fluorophor approach that exploits fluorescence resonance energy transfer (FRET), e.g., LightCycler™ hybridization probes, where two oligonucleotide probes anneal to the amplicon (e.g. U.S. Pat. No. 6,174,670; and US Patent Application No. 20050042618 and references cited therein). The oligonucleotides are labeled with fluorophors and designed to hybridize in a head-to-tail orientation with the fluorophors separated at a distance that is compatible with efficient energy transfer. The FRET signal is seen only when two specific hybridization events occur. A variation of this method has also been employed where a primer internally labeled with a reporter dye serves as a counterpart of one of the detection probes (von Ahsen, et al. Clin. Chem. 46:156-161, 2000). In this variation, the fluorescent dye labeling the primer is incorporated directly into the strand formed by polymerase elongation. The position of the dye on the primer is crucial, as it must be in close enough proximation such that FRET can occur. Further, the probe must hybridize with the strand that is generated by extension of the dye-labeled oligonucleotide to produce a FRET signal.

The current invention provides a new method of using melting curve analysis to identify polymorphisms. The invention employs an oligonucleotide probe labeled with a quenching moiety and a fluorescent-labeled primer that serves as one of the primers in an amplification reaction. Melting curve analysis is employed to detect changes in target nucleic acid sequences.

### BRIEF SUMMARY OF THE INVENTION

In one aspect, the invention provides a method of detecting at least one polymorphic site, the method comprising: (i) amplifying a target nucleic acid sequence comprising the at least one polymorphic site in an amplification reaction that comprises a primer pair that amplifies the target sequence, wherein one of the primers of the primer pair is labeled; (ii) determining the melting point of the amplified reaction in a melting analysis comprising the amplified product and a labeled probe that specifically hybridizes to the nucleic acid target sequence that is amplified by the primer pair, wherein the label on the probe interacts with the label on the primer of (i) such that signal quenching occurs when the labeled primer and labeled probe are in close proximity; and detecting a change in the melting profile in comparison to a control melting profile, thereby detecting the presence of the polymorphic site. In preferred embodiments, the label on the primer is a fluorescent label and the label on the probe is a quencher that quenches the fluorescent label.

In some embodiments, the label is located within 5 nucleotides of the 3′ end of the primer.

The probe can be present during the amplification of the target nucleic acid sequence. In alternative embodiments, the probe is added after the repeated cycles of the amplification reaction and prior to the reannealing step of the melt curve analysis.

The label on the probe can be positioned anywhere in the probe such that it is within the functioning radius of the primer label. In some embodiments, the label on the probe is within 5 nucleotides of the 3′ end of the probe.

In one embodiment, the probe can comprise a modified base at a position that is polymorphic in the target nucleic acid sequence. The modified base can be, for example, a locked nucleic acid nucleotide. In another embodiment, the probe comprises a modified base at a position that is adjacent to the position that is polymorphic in the target nucleic acid sequence.

In some embodiments, the polymorphic site is present in the middle third of the sequence to which the probe hybridizes. In other embodiments, the polymorphic site is adjacent to the sequence to which the probe hybridizes.

In some embodiments, the probe is often from about 10 to about 50 nucleotides in length. Typically, the probe has a higher melting temperature (Tm) than the primers.

In some methods of the invention, the target nucleic acid sequence that is amplified in the amplification reaction comprises more than one polymorphic site. Thus, in some embodiments, the second polymorphic site in the target nucleic acid can be present in the sequence to which the probe hybridizes.

In particular embodiments of the invention, amplification is performed in a multiplex reaction comprising a second primer pair that has one labeled primer, wherein the label is different from the label on the primer in the first primer pair; and a second probe to a second polymorphic site, wherein the second probe is labeled with a quencher that interacts with the label on the labeled primer of the second pair to quench the label signal when the labeled primer of the second pair and the second labeled probe are in close proximity. The method further comprises determining the melting curve profile of the second amplicon, where detecting a shift in the second amplicon is indicative of the presence of the second polymorphic site.

The invention also provides a method of detecting at least two polymorphic sites that are present in an amplicon amplified by a single primer set, the method comprising: (i) amplifying the target nucleic acid sequence with a primer pair having one fluorescent label on the forward primer and a second fluorescent label on the reverse primer; (ii) determining the melting point of the amplified reaction in a melting analysis comprising:

the amplified product;

one probe that specifically hybridizes to one of the polymorphic sites, where the probe is labeled with a quencher that quenches the label on the forward primer when in close proximity;

a second probe that specifically hybridizes to the second polymorphic site, where the probe is labeled with a quencher that quenches the second label on the reverse primer when in close proximity and hybridizes to the opposing strand relative to the first probe; and (iii) detecting a change in at least one of the melting profiles in comparison to a control melting profile, thereby detecting the presence of at least one polymorphic site. In some embodiments, the quencher on the first and the second probes is the same. In other embodiments, the quencher is different.

The sequence targeted by the second probe can overlap the sequence targeted by the first probe. In some embodiments, the two polymorphic sites are separated by about 10 nucleotides or less.

In another aspect, the invention provides amplification reaction mixtures. In one embodiment, the amplification reaction mixture comprises a primer pair that amplifies a target nucleic acid sequence where one primer is labeled with a fluorescent label and the other primer is unlabeled; and a probe that hybridizes to the target nucleic acid sequence where the probe is labeled with a quencher that quenches the fluorescent label when in close proximity.

In another embodiment, the amplification reaction comprises: i) a primer pair that amplifies a target nucleic acid sequence comprising at least two polymorphic sites, where one primer is labeled with a first fluorescent label and the opposing primer is labeled with a second fluorescent label different from the first; ii) a probe that is labeled with a quencher that quenches the first fluorescent label, where the probe hybridizes to a subsequence that contains the first polymorphic site; and iii) a probe that is labeled with a quencher that quenches the second fluorescent label, where the probe hybridizes to a subsequence that contains the second polymorphic site. In some embodiment, the quencher on the first and second probe is the same.

In another aspect, the invention provides kits. In one embodiment, the kit comprises a primer pair that amplifies a target sequence where one primer is labeled with a fluorescent label and the other primer is unlabeled; and a probe that hybridizes to the target nucleic acid sequence amplified by the primer pair, where the probe is labeled with a quencher that quenches the fluorescent label. In another embodiment, the kit comprises: a primer pair that amplifies a target sequence comprising at least a first and a second polymorphic site, where one primer is labeled with a first fluorescent label and the other primer is labeled with a second fluorescent labeled different from the first; a first probe that hybridizes to a subsequence of the target nucleic acid sequence amplified by the primer pair where the subsequence comprises the first polymorphic site, where the probe is labeled with a quencher that quenches the first fluorescent label; and a second probe that hybridizes to a subsequence of the target nucleic acid sequence amplified by the primer pair where the subsequence comprises the second polymorphic site, where the probe is labeled with a quencher that quenches the second fluorescent label. In another embodiment, the invention provides a kit comprising two primer pairs that amplify two target sequences where one primer of the first primer pair that amplifies the first target sequence is labeled with a first fluorescent label and the other primer is unlabeled; and one primer of the second pair is labeled with a second fluorescent label different from the first label; and one probe to the first target sequence, where the probe is labeled with a quencher that quenches the first fluorescent label when in close proximity; and a second probe to the second target sequence, where the second probe is labeled with a quencher that quenches the second fluorescent label when in close proximity.

### BRIEF DESCRIPTION OF THE DRAWINGS

FIG. 1 provides a schematic of a primer and probe arrangement for analysis of a single SNP site.

FIG. 2 provides a schematic of fluorescent profiles of matched and mismatched sequences with a reference target.

FIG. 3 depicts a probe and primer arrangement for analysis of two SNP sites with a single probe.

FIG. 4 shows probe and primer arrangements of independent melt curve analysis of two SNP sites within the same amplicon.

### DETAILED DESCRIPTION OF THE INVENTION

The term “amplification reaction” refers to any in vitro means for multiplying the copies of a target sequence of nucleic acid.

“Amplifying” refers to a step of submitting a solution to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid.

The term “amplification reaction mixture” refers to an aqueous solution comprising the various reagents used to amplify a target nucleic acid. These include, but are not limited to, components such as enzymes, aqueous buffers, salts, amplification primers, target nucleic acid, nucleoside triphosphates, detergents, molecular spacers, modified nucleotides, and the like. Depending upon the context, the mixture can be either a complete or incomplete amplification reaction mixture

“Polymerase chain reaction” or “PCR” refers to a method whereby a specific segment or subsequence of a target double-stranded DNA, is amplified in a geometric progression. PCR is well known to those of skill in the art; see, e.g., U.S. Pat. Nos. 4,683,195 and 4,683,202; and PCR Protocols: A Guide to Methods and Applications, Innis et al., eds, 1990; Sambrook and Russell, MOLECULAR CLONING, A LABORATORY MANUAL (3rd ed. 2001); and CURRENT PROTOCOLS IN MOLECULAR BIOLOGY (Ausubel et al., eds., John Wiley & Sons, Inc. 1994-1997, 2001 version).

A “primer” refers to a polynucleotide sequence that hybridizes to a sequence on a target nucleic acid template and serves as a point of initiation of nucleic acid synthesis. In the context of the invention, a primer is a component in an amplification reaction that participates as a member of a primer pair in the amplification of the target nucleic acid. Primers can be of a variety of lengths and are often less than 50 nucleotides in length, for example 12-25 nucleotides, in length. The length and sequences of primers for use in PCR can be designed based on principles known to those of skill in the art, see, e.g., Innis et al., supra.

A “probe” refers to a polynucleotide sequence that is capable of hybridization to a target polynucleotide sequence of interest and allows for the specific detecting of the polynucleotide sequence of choice. A “probe” in the context of this invention comprises a polynucleotide linked to a quenching reagent, thereby allowing for the detection of these reagents. Thus a “probe” is also referred to herein as a “q-probe”. A “probe” can also include other components, e.g., peptides (as in peptide nucleic acid (PNA)), modified bases, or a spacer that preserves the orientation of the probe sequence.

“Close proximity” as used in the context of this invention with regard to the positions of a fluorphor/quencher pair will vary depending upon the particular fluorophor and quencher. “Close proximity” refers to a distance between a fluorophor and a quencher at which the quencher reduces the fluorescent intensity of the fluorophor. A fluorophor and a quencher are considered to be in “close proximity” when there is a reduction of fluorescence intensity upon binding of the specific probe to the target nucleic acid sequence in the presence of the hybridized primer labeled with the fluorophor. A reduction in fluorescent intensity is typically achieved when the relative fluorescence intensity at the calculated starting point (i.e., at the origin or lowest temperature in the melting curve) is reduced by at least 10%, preferably 50%, in comparison to the peak intensity from the amplified product when compared to a reference un-amplified curve.

A “quencher” as used herein, interacts with a label, e.g., a fluorophor, such that the quencher absorbs the label emission without emitting light.

A “mismatched nucleotide” or a “mismatch” refers to a nucleotide that is not complementary by Watson-Crick pairing to the target sequence at that position.

A “control melting profile” as used herein refers to a reference melting curve generated by a known sequence. The sequence may be a “wild-type” sequence, but is not limited to being a wild-type sequence. The reference can also be an artificial construct that shows melting profile differences between the reference and other sequence changes or alterations.

The term “subsequence” refers to a sequence of nucleotides that are contiguous within a longer sequence, but does not include all of the nucleotides of the longer sequence.

A “target” or “target nucleic acid sequence” refers to a single or double stranded polynucleotide sequence sought to be amplified in an amplification reaction. Two target sequences are different if they comprise non-identical polynucleotide sequences and/or one sequence contains a modified base relative to the other. The target nucleic acid sequence is typically amplified by a primer set in an amplification reaction.

A “temperature profile” refers to the temperature and lengths of time of the denaturation, annealing and/or extension steps of a PCR reaction. A temperature profile for a PCR reaction typically consists of 10 to 60 repetitions of similar or identical shorter temperature profiles; each of these shorter profiles typically define a two step or three-step PCR reaction. Selection of a “temperature profile” is based on various considerations known to those of skill in the art, see, e.g., Innis et al., supra.

A “melting profile” refers to the change in the amount of double-stranded vs. singled-stranded nucleic acid during a melting curve analysis. The melting profile reflects the sequences present in the target nucleic acid.

A “template” refers to a double stranded polynucleotide sequence that comprises the target polynucleotide to be amplified, flanked by primer hybridization sites. Thus, a “target template” comprises the target polynucleotide sequence and the flanking hybridization sites for a 5′ primer and a 3′ primer.

An amplicon refers to a nucleic acid sequence that is amplified by a primer pair during an amplification reaction.

“Multiplex amplification” refers to amplification of multiple polynucleotide fragments in the same reaction (see, e.g., PCR PRIMER, A LABORATORY MANUAL (Dieffenbach, ed. 1995) Cold Spring Harbor Press, pages 157-171).

A “polymorphism” in the context of this application is an allelic variant. Polymorphisms can include single nucleotide polymorphisms as well as simple sequence length polymorphisms. A polymorphism can be due to one or more nucleotide substitutions at one allele in comparison to another allele or can be due to an insertion or deletion. “Polymorphism” as used herein refers to any sequence variation and thus includes those polymorphisms that are silent as well as those that have a phenotypic effect, e.g., are mutations. Thus, “polymorphism” may include single base changes, mutations, sequence insertions/deletions/inversions as long as they occur within probe target region.

Introduction

The present invention provides a new method of determining the presence of nucleic acid sequence variations in a target nucleic acid sequence. The method employs a labeled primer, an unlabeled opposing primer, i.e., the second primer in the primer pair used for a polymerase chain reaction to amplify the target nucleic acid sequence, and a probe that is labeled with a quenching molecule. The target nucleic acid is amplified in an amplification reaction using the primer pair. The amplified products are then subjected to melt curve analysis in the presence of the quenching probe. In the melt curve analysis, fluorescence is monitored and the amplified products are gradually heated. An increase in fluorescense is obtained when the probe melts off of the target nucleic acid sequence. The presence of a nucleotide change in the target nucleic acid sequence to which the oligonucleotide probe hybridizes changes the melting curve profile relative to a sequence that does not harbor the nucleotide change, thereby determining the presence of a sequence variation in the targeted region of the nucleic acid of interest.

Oligonucleotide Probes and Primers

Oligonucleotide primers and probes for use in the methods of the invention can be prepared using any suitable method, such as, for example, methods using phosphotriesters and phosphodiesters well known to those skilled in the art. In some embodiments, one or more phosphorothioate linkages may be included in the probe. The oligonucleotide can also be modified at the base moiety, sugar moiety, or phosphate backbone with minor groove binders, intercalating agents and the like.

The primers for the amplification reactions are designed according to known algorithms. The primers are designed to hybridize to sequences that flank the target nucleic acid, i.e., the region of the nucleic acid that contains the sequence to be analyzed for the presence of one or more sequence variations. Often, commercially available or custom software will use algorithms to design primers such that the annealing temperatures are close to melting temperature. Amplification primers are usually at least 12 bases, more often about 15, 18, or 20 bases in length. Primers are typically designed so that all primers participating in a particular reaction have melting temperatures that are within 5° C., and most preferably within 2° C. of each other. Primers are further designed to avoid priming on themselves or each other. Primer concentration should be sufficient to bind to the amount of target sequences that are amplified so as to provide an accurate assessment of the quantity of amplified sequence. Those of skill in the art will recognize that the amount of concentration of primer will vary according to the binding affinity of the primers as well as the quantity of sequence to be bound. Typical primer concentrations will range from 0.01 μM to 1.0 μM. Primer concentration is also considered with regard to probe concentration. Typically, the probe concentration is in excess of both primers.

The amplification reactions are incubated under conditions in which the primers hybridize to the target sequence template and are extended by a polymerase. As appreciated by those of skill in the art, such reaction conditions may vary, depending on the target nucleic acid of interest and the composition of the primer. The amplification reaction cycle conditions are selected so that the primers hybridize specifically to the target template sequence and are extended. Primers that hybridize specifically to a target template amplify the target sequence preferentially in comparison to other nucleic acids that may be present in the sample that is analyzed. A temperature profile for the cycles of amplification can be determined empirically.

One of the primers in a primer pair that amplifies the nucleic acid region of interest is labeled with a fluorophor that interacts with a quencher such that the quencher absorbs the fluorophor emission without emitting light. The fluorophor is typically positioned internally on the primer, e.g., three to six bases from the 3′ end of the primer, for ease of synthesis. In some embodiments, the fluorphor can be at the 5′ end of the primer, so long as it is within effective range for quenching by the quencher on the probe.

Hybridization Probes

The probe oligonucleotides for use in the invention can be any suitable size, and are often in the range of from about 6 to about 100 nucleotides, more often from about 6 to about 80 nucleotides and frequently from about 10 to about 40 nucleotides. The precise sequence and length of an oligonucleotide probe depends in part on the nature of the target polynucleotide to which it binds. The binding location and length may be varied to achieve appropriate annealing and melting properties for a particular embodiment. For example, the length will depend on the resolution required, the desired melting temperature for the probe and the number of modified or substituted bases incorporated within the probe. Typical melt curves show a lower melting temperature for mismatches than perfect matches. Guidance for making such design choices can be found in many art recognized references. The probe is complementary to the extended product of the labeled primer.

There are a number of considerations in selecting primers and probe sets for use in the invention. For example, typically the probe has a higher Tm than the labeled primer because the labeled primer is part of the extension product and cannot melt off. The probe therefore can be designed to have at least a slightly higher Tm so that it will “out-compete” the nascent strand hybridization during the re-annealing step performed for the melting curve analysis. Other considerations in optimizing primer and probe parameters include the polymerase that is used in the amplification assay. For example, when using a polymerase that has exonuclease activity, some of the probe will be digested; thus, the probe is present in a sufficiently excess amount so as not to affect the analysis. When using displacing polymerases, the probe often does not need to be in as great an excess.

Detection of Sequence Variants

The methods of the invention are typically used to detect sequence polymorphisms, e.g., single base substitutions, in a nucleic acid sequence of interest. The sample nucleic acid to be evaluated can be RNA or DNA. In instances where the nucleic acid sample is RNA, a reverse transcription step is typically employed.

The nucleic acid sequence to be targeted by the hybridization probe is a region of interest that is suspected of containing a sequence variation. Sequence variations can comprise include nucleotide substitutions, deletions or additions. Characteristic melting profiles for probes and target sequences will distinguish the various polymorphisms that occur. The methods of the invention provide for the identification of more than one polymorphism within the region covered by the probe. For example, some SNPs occur very close together, e.g., within 10 bases of each other.

The probes for use in the reaction therefore target the polymorphic region of the nucleic acid of interest. In preferred embodiments, the probe is designed to include the polymorphic region, i.e., the probe comprises the polymorphic site. For example, a probe can be designed to detect an allele that is polymorphic at a particular site. The probe is typically designed such that it comprises the position that is polymorphic, i.e., it hybridizes to the target nucleic acid sequence at the polymorphic site. The probe can be an exact match to the target nucleic acid or can have a mismatch. When there is a base mismatch, the melting point is lower in comparison to when the probe matches the target nucleotide at the polymorphic position. The polymorphic site can be at any position in the probe. In some embodiments, it may be desirable to select a target binding site such that the polymorphic site is in the middle third of the probe in order to enhance overall stability.

In other embodiments, the probe may not hybridize to the subsequence of the amplicon that contains the sequence variation to be detected, but can still detect a change in melting profile due to the presence of the sequence variation. For example, the probe may hybridize to a position adjacent to the variant site such that the presence of the sequence variation alters the Tm of the probe. The probe hybridizes to a site that is sufficiently close to the positions of the labeled primer to quench the primer. During the melt curve analysis, the presence of a sequence variation is detected by detecting a change in the melting curve due to a change in the Tm of the probe that results from the sequence variation.

Often, the probe is present in excess relative to the primer. However, as appreciated by those in the art, the amount of probe and primer can be empirically determined by performing trial melting curve analyses to maximize discrimination between variant alleles.

The probe can comprises modified bases, such as locked nucleic acids (LNAs), peptide-nucleic acids, or other base substitution. Typically, such substitute bases are employed to enhance discrimination of Tm differences between matched and mismatched sequences.

In some embodiments, a locked nucleic acid modification is employed. LNAs refer to bicyclic and tricyclic nucleoside and nucleotide analogs, and the oligonucleotides that contain such analogs. The basic structural and functional characteristics of LNAs and related analogs are disclosed in various publications and patents, including WO 99/14226, WO 00/56748, WO 00/66604, WO 98/39352, U.S. Pat. No. 6,043,060, and U.S. Pat. No. 6,268,490. Where locked nucleic acids are employed in the quencher probe, the quencher probes can be shortened to increase the annealing temperature. LNA can also be placed opposite the polymorphic site to maximize the mismatch and thereby increase the melting temperature differences to discriminate between matches and mismatches.

Where the probe is present during the amplification reaction, modified bases included in the probe are often positioned so that they do not halt primer extension during amplification. In applications in which a modified base stops extension, the modified base is often positioned toward the 3′ end of the probe.

Labels

The hybridization probe is typically labeled with a fluorescent molecule that can be quenced by a quenching molecule as described herein. Fluorescent labels are well known in the art. Examples of fluorescence labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Cyanine dyes (Cy3, Cy5, Cy3.5, Cy5.5), Dansyl, Dapoxyl, Dialkylaminocoumarin, 4′, 5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, and Texas Red-X.

The assays of the invention employ a quencher in conjunction with the fluorescent moiety. A quencher includes any moiety that is capable of absorbing the energy of an excited fluorescent label when located in close proximity to the fluorescent label and capable of dissipating that energy without the emission of visible light. Examples of quenchers include, but are not limited to, DABCYL (4-(4′-dimethylaminophenylazo)benzoic acid)succinimidyl ester, diarylrhodamine carboxylic acid, succinimidyl ester (QSY-7), and 4′,5′-dinitrofluorescein carboxylic acid, succinimidyl ester (QSY-33) (all available from Molecular Probes, now part of Invitrogen), quencherl (Q1; available from Epoch), or Iowa Black™ quenchers (Integrated DNA Technologies), and “Black hole quenchers” BHQ-1, BHQ-2, and BHQ-3 (available form BioSearch, Inc.).

The quencher label present in the probe may be present at the end, but it need not be. For example, a quencher moiety may be at least 1, 2, 3, 4, 5, 6, 7, 8, 9, 10, or more nucleotides away from the end of the probe, so long as the distance allows the quencher to interact with the fluorescent label on the primer when the probe (and primer) are hybridized to the target nucleic acid. Typically, the quencher will be within 30 nucleotides of the primer, but this distance is not required. The maximum distance will vary depending on the radius of the quenching action. A fluorophor and a quencher are considered to interact when there is a reduction of fluorescence intensity upon binding of the specific probe to the target nucleic acid sequence in the presence of the hybridized primer labeled with the fluorophor. The reduction in fluorescent intensity is typically at least 10% relative fluorescence intensity between the origin or lowest temperature in the melt curve, and peak intensity for the amplified product when compared to a reference un-amplified curve. For example, if the peak relative fluorescence intensity.(RFI) is 980 units at 57° C., then the baseline for the start of the curve is typically about 882 RFI or less.

Base-linked fluors and quenchers are readily available in the art. They can be obtained, for example, from Life Technologies (Gaithersburg, Md.), Sigma-Genosys (The Woodlands, Tex.), Genset Corp. (La Jolla, Calif.), Synthetic Genetics (San Diego, Calif.), or Biosearch Technologies (Novato, Calif.). In some cases, base-linked fluors or quenchers are incorporated into the oligonucleotides by post-synthesis modification of oligonucleotides that were synthesized with reactive groups linked to bases. The fluor or quencher can be attached to the 3′ OH of the sugar or the base.

Practical guidance is readily available in the literature for selecting appropriate fluors or quenchers for particular primers or probes, as exemplified by the following references: Pesce et al., Eds., Fluorescence Spectroscopy (Marcel Dekker, New York, 1971); White et al., Fluorescence Analysis: A Practical Approach (Marcel Dekker, New York, 1970). The literature also includes references providing exhaustive lists of fluorescent and chromogenic molecules and their relevant optical properties for choosing reporter-quencher pairs (see, for example, Berlman, Handbook of Fluorescence Spectra of Aromatic Molecules, 2nd Edition (Academic Press, New York, 1971); Griffiths, Colour and Constitution of Organic Molecules (Academic Press, New York, 1976); Bishop, Ed., Indicators (Pergamon Press, Oxford, 1972); Haugland, Handbook of Fluorescent Probes and Research Chemicals (Molecular Probes, Eugene, 1992) Pringsheim, Fluorescence and Phosphorescence (Interscience Publishers, New York, 1949). Further, the literature provides ample guidance for derivatizing reporter and quencher molecules for covalent attachment via common reactive groups that can be added to an oligonucleotide (see, e.g., Haugland (supra); U.S. Pat. No. 3,996,345; and U.S. Pat. No. 4,351,760).

Primer labeling can be at any position within the primer sequence as long as it satisfies two conditions: primer extension can occur and the fluor is within the effective quenching range of the quenching probe.

Melting curve analysis is performed using standard techniques. In this method, when a probe labeled with a quencher hybridizes to an allele, the quencher quenches signal from the primer. Fluorescence is monitored while gradually increasing the temperature of the amplification reaction products. As the probe melts off during heating for the melting analysis, the quencher no longer quenches the label on the primer, and a fluorescent signal is generated. The mid-point of the melting curve is determined and the corresponding temperature is measured as Tₘ. When a probe hybridizes to a mismatched allele, the probe will be melted away at a lower temperature, thereby generating a change in the melting curve.

The hybridization probes may already be present during the amplification reaction or added subsequently. Typically, in the melt curve analysis amplified products generated in a reaction mixture comprising a labeled primer are heated following one or more desired cycles of amplification, cooled, e.g., to 40° C., and slowly heated, typically to a temperature of about 95° C. The oligonucleotide probe comprising the quencher is conveniently added at the beginning of the amplification process, but may be added, e.g., at the heating stage following amplification before the melt analysis is performed. As noted above, the probe is typically designed to have a higher Tm than the primer sequences. It therefore hybridizes to the target sequence before the primers. As the forward primer is extended during the amplification reaction in reactions that employ a DNA polymerase having 5′ to 3′ exonuclease activity, the 5′ to 3′ exonuclease activity will digest the probe and the probe will be released from the target DNA strand. In this circumstance, there should be an excess of probe vs. forward primer. The amount of excess is generally empirically determined, but can be, e.g., twice the concentration of the reverse (fluor-labeled) primer. If the forward primer is labeled, the probe will hybridize to the extension product and only need to be present in clear excess of that primer. As one of skill in the art understands, the amounts of probe and primers can be empirically determined to optimize the reactions.

The shape and position of a melting curve is a function of GC/AT ratio, length, and sequence, and can be used to differentiate amplification products separated by a small increment in melting temperature. The melting profile obtained from a test reaction is typically compared to a control melting profile. The control melting profile is a reference melting curve generated by a known sequence. The sequences, can be, e.g., a wild-type sequence, but can also be another known sequence, such as an artificial construct that maximizes melting profile differences between the reference and other sequence changers.

Detection of Multiple Polymorphic Sites

Analysis of melting curves can be used to distinguish the presence of more than one target nucleic acid sequences in amplification reactions, including multiplex amplification reactions.

The q-probe reactions of the invention can be readily used in a multiplex analysis, such as evaluating multiple variant sequence sites within a single reaction vessel. For example, one SNP in a target nucleic acid sequence of interest can correspond to one fluor (i.e., the target nucleic acid that includes the SNP site is amplified with a primer pair where one of the primers is labeled with a particular fluor) and a second SNP can correspond to a second fluor (i.e., the target nucleic acid that has the second SNP site is amplified with a primer pair where one of the primers is labeled with a second fluor). Probes that detect the two SNP sites are labeled with quencher. The multiple SNPs are detected by monitoring the fluorescence corresponding to the two labeled primers.

Multiplex reactions can be employed to evaluate multiple polymorphic sites of interest that occur at non-overlapping target regions. In other embodiments, multiple polymorphic sites that occur in a region amplified by the same primer pair can be evaluated. For example, two target regions can be assayed at the same time with two sets of primers that amplify non-overlapping target regions. Each primer pair can contain a labeled and unlabeled primer. The label on the labeled primer of each primer set is matched with an appropriate quencher. Two different fluors are used and monitored with each fluor interrogating a single region.

In alternative embodiments, two different regions within a target nucleic acid amplicon that is amplified by a single primer pair are assayed. For example, if two SNPs are too far apart for a single probe to interrogate both SNPs, or if it is desirable to separately analyze each SNP, two probes, one targeted to one SNP and the other to the second SNP, can be used with a common set of primers. In this configuration, each primer in the primer pair is labeled, each with a different fluor. The two q-probes hybridize to opposite strands. The quencher that is present on the two probes may or may not be different depending on the compatibility with the appropriate fluor.

Kits

In another aspect, the invention also provides kits for performing analysis of target nucleic acids for the presence of one or more polymorphisms. Such kits typically comprise a primer pair that amplifies the nucleic acid sequence of interest where one of the primers is labeled with a fluor and the other primer is not labeled; and a q-probe that is labeled with a quencher that quenches the fluor on the primer. The kit also typically contains instructions for using the primers/probe. Other components, e.g., PCR reagents, can optionally be included in the kit.

A kit can also comprise components for multiplex PCR. For example, the kit may comprise more than one primer set, each of which has one primer that is labeled with a fluor and one primer that is not labeled. The fluor for each primer set is different. Such a kit also comprises multiple q-probes corresponding to the various polymorphic sites to be analyzed in the multiplex reaction. The q-probes can be labeled with the same quencher or different quenchers, depending on the fluors that label the primers.

In another embodiment, e.g., for detecting the presence of two polymorphic sites in a single amplicon, a kit can comprise a primer pair for amplifying the region of interest where each of the primers is labeled with a different fluor, and two q-probes where each q-probe detects one of the polymorphic sites present in the single amplicon.

The invention thus also comprises a reaction mixture comprising a primer pair that amplifies a target sequence where one primer is labeled with a fluorescent label and the other primer is unlabeled; and a probe that hybridizes to the target nucleic acid sequence where the probe is labeled with a quencher that quenches the fluorescent label on the primer when in close proximity.

### EXAMPLES

The following examples are provided for duplex DNA. However, an RNA nucleic acid samples can also be evaluated in the same manner.

### Example 1

### Detection of a Single Variant Site

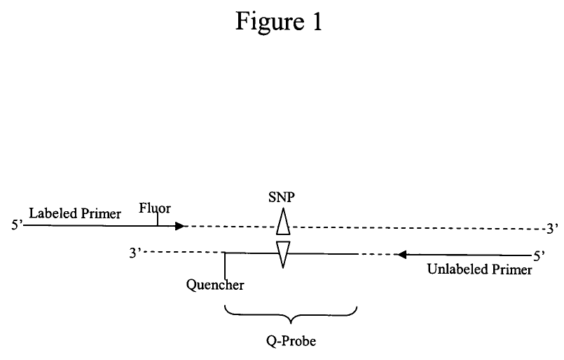

An exemplary Q-melt analysis is performed as follows. FIG. 1 shows the configuration of the primer and probe for the analysis. As primer 1 (labeled with a fluorophor) is extended during the amplification reaction, it extends beyond the SNP site. The Q probe contains a quencher that quenches the fluor. The Q probe targets, i.e., is designed to hybridize to, the subsequence containing the SNP site. During PCR, the Q probe will be cleaved by the 5′ to 3′ exonuclease activity of a DNA polymerase that extends primer 2, the unlabeled primer. Thus, in this example, an excess of Q probe is included in the reaction mixture. A displacing polymerase enzyme may also be used that would not cleave the Q probe.

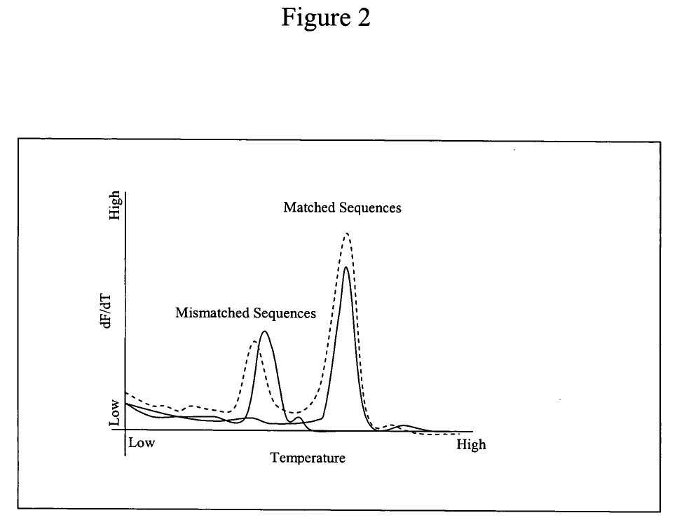

After the amplification cycle(s), the reaction mixture containing the amplified DNA, primer, and probe is subjected to a temperature gradient. Starting at a low temperature, e.g., 50° C., all or most of the labeled primer is incorporated in the amplified DNA and is therefore is near, i.e., at a distance that quenches, the hybridized Q probe. Thus, there is little overall fluorescence. As the temperature is increased, the Q probe melts off of the extended target strand of DNA and there is an increase in fluorescence. The melting temperature is characteristic of a base match or mismatch. The presence of a mismatched nucleotide in the target nucleic acid at the targeted polymorphic site shifts the melting curve such that the mismatched sequence has a lower Tm. A schematic of melting curves showing a shift that results from a mismatch is provided in FIG. 2.

In some embodiments, the Q probe spans more than one polymorphic site. Thus, characteristic melting profiles can be obtained for either or both polymorphic sites.

### Example 2

### Analysis of Multiple Sites

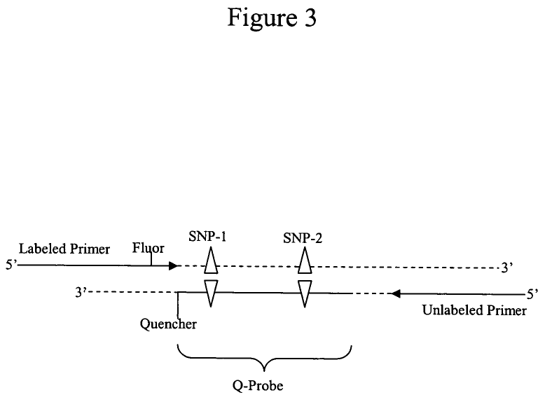

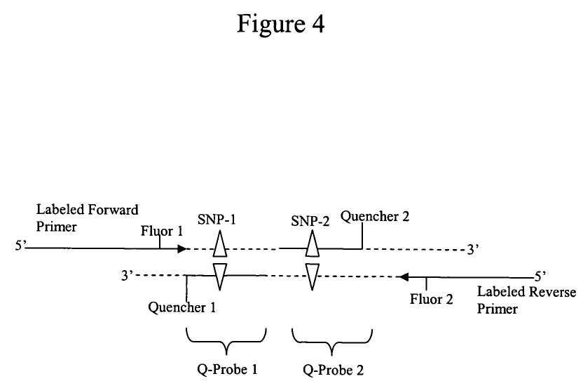

The HFE gene, which is involved in hemochromatosis, has three SNP sites of interest: C282Y, H63D, and S65C. As noted in Example 1, the sites in close proximity (H63D and S65C) can be evaluated using a single probe. In many cases, however, it is desirable to examine the SNP at S65C independently of the nearby H63D. The schematic in FIG. 3 shows a probe and primer arrangement for analysis of two adjacent SNP sites using a single Q-probe. The scheme diagrammed in FIG. 4 shows independent Q-melt probe analysis for SNP detection.

In an analysis in which independent Q-probes are employed to detect two SNP sites that are amplified by the same primer set, one fluorophor for each SNP probe is employed. The melting curve is used to determine the presence of the various polymorphisms (wt or mutant). FIG. 4 depicts an arrangement for such an analysis. The Q-probes are designed to hybridize to opposite strands. The probes may overlap each other, e.g., if necessary to enhance stability or provide the melting curve resolution need. This configuration is often employed when the SNP sites are close to each other, for example, where there is less than about 10 bases separation. In an example evaluating the H63D and S65C SNP sites in the Hereditary Hemochromatosis gene, the SNPs are separated by 5 bases and probes against opposite strands are used.

SNP analysis using two Q-probes within a region amplified by the same primer set requires efficient hybridization of the probes to the SNP target regions. Modified bases can be used, for example, to adjust the melting temperatures to within a functional range of the assay. In this example, both probes typically have a higher Tm than the primers and have Tm's within a few, e.g., 5° C. of each other.

All publications, patents, accession numbers, and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of detecting a polymorphic site, the method comprising:
(i) amplifying a target nucleic acid sequence comprising at least one polymorphic site in an amplification reaction that comprises a primer pair that amplifies the target sequence, wherein at least one of the primers of the primer pair is labeled;
(ii) determining the melting point of the amplified reaction in a melting analysis comprising the amplified product and a labeled probe that specifically hybridizes to the nucleic acid target sequence that is amplified by the primer pair, wherein the label on the probe interacts with the label on the primer of (i) such that signal quenching occurs when the labeled primer and labeled probe are in close proximity; and
detecting a change in the melting profile in comparison to a control melting profile, thereby detecting the presence of a polymorphic site.

2. The method of claim 1, wherein the label on the labeled primer is a fluorescent label and the label on the probe is a quencher that quenches the fluorescent label.

3. The method of claim 1, wherein the label on the primer is within 5 nucleotides of the 3′ end of the primer.

4. The method of claim 1, wherein the probe is present during the step of amplifying the target sequence.

5. The method of claim 1, wherein the label on the probe is within 5 nucleotides of the 3′ end of the probe.

6. The method of claim 1, wherein the probe comprises a modified base at a position that is polymorphic in the target nucleic acid sequence.

7. The method of claim 6, wherein the modified base is a locked nucleic acid nucleotide.

8. The method of claim 1, wherein the probe comprises a modified base at a position that is adjacent to the position that is polymorphic in the target nucleic acid sequence.

9. The method of claim 1, wherein at least one polymorphic site in the target nucleic acid is in the sequence to which the probe hybridizes.

10. The method of claim 9, wherein a second polymorphic site in the target nucleic acid is in the sequence to which the probe hybridizes.

11. The method of claim 9, wherein the polymorphic site is present in the middle third of the sequence to which the probe hybridizes.

12. The method of claim 1, wherein the probe is from about 10 to about 50 nucleotides in length.

13. The method of claim 1, wherein the probe has a higher Tm than the primers.

14. The method of claim 1, wherein the amplification is performed in a multiplex amplification reaction comprising a second primer pair that has one labeled primer, wherein the label is different from the label on the primer in the first primer pair; and a second probe that has a label, wherein the label on the probe interacts with the label on the labeled primer of the second pair to quench the label signal when the labeled primer of the second pair and the second labeled probe are in close proximity; and
the method further comprises determining the melting profile for the second probe.

15. The method of claim 1, further comprising detecting a change in a melting profile using a second probe labeled with a quencher that targets a second polymorphic site present in the target nucleic acid amplified by the primer pair, where the second primer of the primer pair is labeled with a fluorescent label that is different from the label on the first labeled primer of the primer pair and is quenched by the quencher on the second probe.

16. A method of detecting at least two polymorphic sites that are present in an amplicon amplified by a single primer set, the method comprising:
(i) amplifying the target nucleic acid sequence with a primer pair having one fluorescent label on the forward primer and a second fluorescent label on the reverse primer;
(ii) determining the melting point of the amplified reaction in a melting analysis comprising:
the amplified product;
one probe that specifically hybridizes to one of the polymorphic sites, where the probe is labeled with a quencher that quenches the label on the forward primer when in close proximity;
a second probe that specifically hybridizes to the second polymorphic site, where the probe is labeled with a quencher that quenches the second label on the reverse primer when in close proximity and hybridizes to the opposing strand relative to the first probe; and
detecting a change in at least one of the melting profiles in comparison to a control melting profile, thereby detecting the presence of at least one polymorphic site.

17. The method of claim 16, wherein the same quencher is used on the first and the second probe.

18. The method of claim 16, wherein the quencher on the first probe is different from the quencher on the second probe.

19. The method of claim 16, where the sequence targeted by the second probe overlaps the sequence targeted by the first probe.

20. The method of claim 16, wherein the two polymorphic sites are separated by about 10 nucleotides or less.

21. An amplification reaction mixture comprising a primer pair that amplifies a target sequence where one primer is labeled with a fluorescent label; and a probe that hybridizes to the target nucleic acid sequence where the probe is labeled with a quencher that quenches the fluorescent label.

22. A kit comprising a primer pair that amplifies a target sequence where one primer is labeled with a fluorescent label and the other primer is unlabeled; and a probe that hybridizes to the target nucleic acid sequence amplified by the primer pair, where the probe is labeled with a quencher that quenches the fluorescent label

23. A kit comprising:
a primer pair that amplifies a target sequence comprising at least a first and a second polymorphic site, where one primer is labeled with a first fluorescent label and the other primer is labeled with a second fluorescent labeled different from the first;
a first probe that hybridizes to a subsequence of the target nucleic acid sequence amplified by the primer pair where the subsequence comprises the first polymorphic site, where the probe is labeled with a quencher that quenches the first fluorescent label; and
a second probe that hybridizes to a subsequence of the target nucleic acid sequence amplified by the primer pair where the subsequence comprises the second polymorphic site, where the probe is labeled with a quencher that quenches the second fluorescent label.

24. A kit comprising two primer pairs that amplify two target sequences where one primer of the first primer pair that amplifies the first target sequence is labeled with a first fluorescent label and the other primer is unlabeled; and one primer of the second pair is labeled with a second fluorescent label different from the first label; and one probe to the first target sequence, where the probe is labeled with a quencher that quenches the first fluorescent label when in close proximity; and a second probe to the second target sequence, where the second probe is labeled with a quencher that quenches the second fluorescent label when in close proximity.
